(19) United States
(12) Patent Application Publication
(10) Pub. No.: US 2009/0137058 A1
(43) Pub. Date: May 28, 2009

(54) CHEMICAL AND BIOLOGICAL DETECTION METHOD AND DEVICE BASED ON MEASUREMENTS OF FLUORESCENCE AND REFLECTIVITY

(75) Inventors: Eugene Barash, Niskayuna, NY (US); Andrew David Pris, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 11/946,342

(22) Filed: Nov. 28, 2007

(51) Int. Cl. G01N 21/64; C12M 1/34

(52) U.S. Cl. 436/172; 422/82.08; 435/288.7

(57) ABSTRACT

A device and method for detecting the presence of one or more analytes, bound directly or indirectly to a binding substrate functionalized with a fluorophore, based on measurements of fluorescence and reflectivity. The device and methods comprise an excitation source that emits light capable of being absorbed by a fluorophore and results in the fluorophore's excitation and emission, a fluorescent probe specific for the analyte that is attached via chemisorption to the binding substrate, a detector, and a processor adapted to determine the quantity of the one or more analytes present, by correlating measurements of reflected and fluorescent light.

### STATEMENT REGARDING FEDERALLY SPONSORED RESEARCH & DEVELOPMENT

This invention was made with Government support under contract number W91 SR-05-C-0003 awarded by the United States Army RDECOM. The Government has certain rights in the invention.

### BACKGROUND

Disclosed herein are devices and methods for quantitative analysis of chemical and biological materials, analytes, immobilized on a binding substrate based on measurements of fluorescence and reflectivity.

Quantitative measurements of analytes using light based biological and chemical probes are well known and include methods that immobilize the probes on solid surfaces such as a thin layer of gold film wherein the probes are exposed to the analytes which are subsequent detected through luminescence, typically fluorescence. However the sensitivity and accuracy of these methods, which involve a photon adsorption/scatter to create this luminescence, are limited largely due to morphology differences across the resulting layered surface that causes variations in specular and diffuse reflectivity. The resulting reflective light may cause a secondary excitation of the bound probe and contribute to additional fluorescence being measured. The angle and wavelength of incident light, the refractive index of the substrate components or surrounding medium, and polarization of the incident light also affect reflectivity. These variations limit device calibration and method reproducibility.

A shortcoming of light based measurements on high-reflectivity metal films in general and as it specifically relates to gold films, is the change in the dielectric behavior of gold in blue light; gold performs as a dielectric rather than a metal for blue to violet light (300 nm to 500 nm). As a result, within this wavelength range, as a physi- or chemi-sorbing molecular species increases its coverage upon the gold surface from sub- to full monolayer coverage, there is a corresponding a reduction in the number of photons reflected from the gold surface. This phenomenon is referred to as the anomalous reflection of gold (hereinafter “AR”). While reduction in gold reflectivity in blue light is desirable in fluorescent spectroscopy, since AR is not a resonance effect, it varies over the entire blue light range. Consequently, the degree of photons reflecting off the gold substrate within this AR phenomenon is dependent upon both the wavelength of light and the coverage of the sorbing molecular species. Importantly this results in secondary fluorescence of species at the gold surface to be dependent upon these same two criteria, wavelength and coverage of sorbing species. The secondary fluorescence intensity is dependent upon the flux of photons into its environment both from the light source as well as those reflected from the surface. If the flux of photons is fluctuating, as a function of wavelength and coverage, then the fluorescence intensity will also depend upon these factors.

An alternative class of gold film based optical probes utilizes a surface plasmon resonance effect (hereinafter “SPR”) in the absence of fluorescently labeled probes. Using SPR probes, light intensity or wavelength changes are measured as a function of the complex refractive index of the proximal samples. These probes are widely used to study biochemical reactions but suffer from relatively low sensitivity, 10⁻³-10⁻⁶ refractive index units (RIU), and high cost. In addition, an analyte's bulk refractive index, which is highly temperature dependent, has a strong affect on SPR accuracy. Therefore the use of SPR probes in quantitative sensing applications requiring measurements over a wide temperature range, such as in DNA annealing and de-annealing, is limited.

This invention is directed to overcome the limitations of existing light based solid support probes as described above. More specifically it relates to a multimode detection method wherein both reflected and fluorescent light are detected sequentially or simultaneously from the sample in such a way as to improve the accuracy of quantification by correcting for changes in surface reflection.

### BRIEF DESCRIPTION

In a first aspect, the invention provides a device for detecting the presence of one or more analytes, bound directly or indirectly to a binding substrate functionalized with a fluorophore, based on measurements of fluorescence and reflectivity. The device comprises an excitation source that emits light capable of being absorbed by a fluorophore and resulting in the fluorophore's excitation and emission, a fluorescent probe specific for the analyte that is attached via chemisorption to the binding substrate, a detector, and a processor adapted to determine the quantity of the one or more analytes present by correlation of measurements of reflected and fluorescent light.

In a second aspect, the invention provides a method for detecting the presence, of one or more analytes bound directly or indirectly to a binding substrate functionalized with a fluorophore. The method comprises illuminating the binding substrate, measuring the fluorescent light and reflected light emitted, and determining whether one or more analytes is bound to the binding substrate by correlating the measurements of emitted fluorescent light and reflected light.

### DRAWINGS

These and other features, aspects, and advantages of the present invention will become better understood when the following detailed description is read with reference to the accompanying drawings in which like characters represent like parts throughout the drawings, wherein:

FIG. 1 is a cross-sectional view of an embodiment of the imaging device of the invention;

FIG. 2 graphically shows a spectral profile of fluorescence emission and reflectivity;

FIG. 3 shows reflectivity spectra as a function of analyte concentration.

FIG. 4 shows real-time reflectivity measurement of dsDNA at 470 nm at normal incidence;

FIG. 5 is a cross-sectional view of another embodiment of the imaging device of the invention comprising a micro-heater;

FIG. 6a shows a DNA melt curve of average fluorescent intensity vs. temperature.

FIG. 6b shows a DNA melt curve of fluorescent signal vs. incubation time.

FIG. 7 illustrates one embodiment of an imaging device with a removable chamber.

### DETAILED DESCRIPTION

As used herein “analyte” refers to any detectable chemical or biological species or moiety or moieties that is of interest. These include peptides, proteins, nucleic acids, oligonucleotides, signaling molecules, prokaryotic or eukaryotic cells, viruses, subcellular organelles, and any other biological and chemical compounds. The term “peptide” refers to oligomers or polymers of any length wherein the constituent monomers are alpha amino acids linked through amide bonds, and encompasses amino acid dimers as well as polypeptides, peptide fragments, peptide analogs, naturally occurring proteins, mutated, variant or chemically modified proteins, fusion proteins, and the like. The amino acids of the peptide molecules may be any of the twenty conventional amino acids, stereoisomers (e.g., D-amino acids) of the conventional amino acids, structural variants of the conventional amino acids, e.g., iso-valine, or non-naturally occurring amino acids such as α,α-disubstituted amino acids, N-alkyl amino acids, β-alanine, naphthylalanine, 3-pyridylalanine, 4-hydroxyproline, O-phosphoserine, N-acetylserine, N-formylmethionine, 3-methylhistidine, 5-hydroxylysine, and norleucine. In addition, the term “peptide” encompasses peptides with posttranslational modifications such as glycosylations, acetylations, phosphorylations, and the like.

The term “oligonucleotide” is used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the oligonucleotide. More particularly, the term includes polydeoxyribonucleotides (containing 2-deoxy-D-ribose), polyribonucleotides (containing D-ribose), any other type of polynucleotide which is an N- or C-glycoside of a purine or pyrimidine base, and other polymers containing normucleotidic backbones, for example, polyamide (e.g., peptide nucleic acids (PNAs)) and polymorpholine (commercially available from the Anti-Virals, Inc., Corvallis, Oregon, as Neugene) polymers, and other synthetic sequence-specific nucleic acid polymers, providing that the polymers contain nucleobases in a configuration that allows for base pairing and base stacking, such as is found in DNA and RNA. There is no intended distinction in length between the terms “polynucleotide”, “oligonucleotide”, “nucleic acid” and “nucleic acid molecule”, and these terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps”, substitution of one or more of the naturally occurring nucleotides with an analog, internucleotide modifications such as, for, example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein “probe” refers to a moiety that possesses specificity to a desired analyte (e.g., peptides, proteins, enzymes, antibodies, chelators, nucleic acids, polymers, or ligands). The probe can be naturally occurring or chemically synthesized. The probe employed may have desired physical, chemical, or biological properties, including, but not limited to, covalent and noncovalent association with peptides, proteins, nucleic acids, signaling molecules, prokaryotic or eukaryotic cells, viruses, subcellular organelles and any other biological and chemical compounds. Probes may also be the ability to affect a biological process (e.g. cell cycle, blood coagulation, cell death, transcription, translation, signal transduction, DNA damage or cleavage, production of radicals, scavenging radicals, etc.), or alter the structure of a biological compound (e.g. crosslinking, proteolytic cleavage, radical damage, etc.).

As used herein “fluorescent probe” refers to a probe complexed with a fluorophore. In one embodiment, the fluorophores are initially bound to the probe, or bound to the probe at least prior to interaction between the probe and the analyte. In an alternative embodiment, the fluorophores (or fluorophore-intercalator complexes) are tethered to the same surface as the probe and are positioned such as to allow the fluorophore to associate with the probe-analyte complexes. In other embodiments, the fluorophores are free in a sample solution with the analyte. When the probe and the analyte interact with each other to form probe-analyte complexes, the fluorophore associates with the surface bound probe-analyte complex.

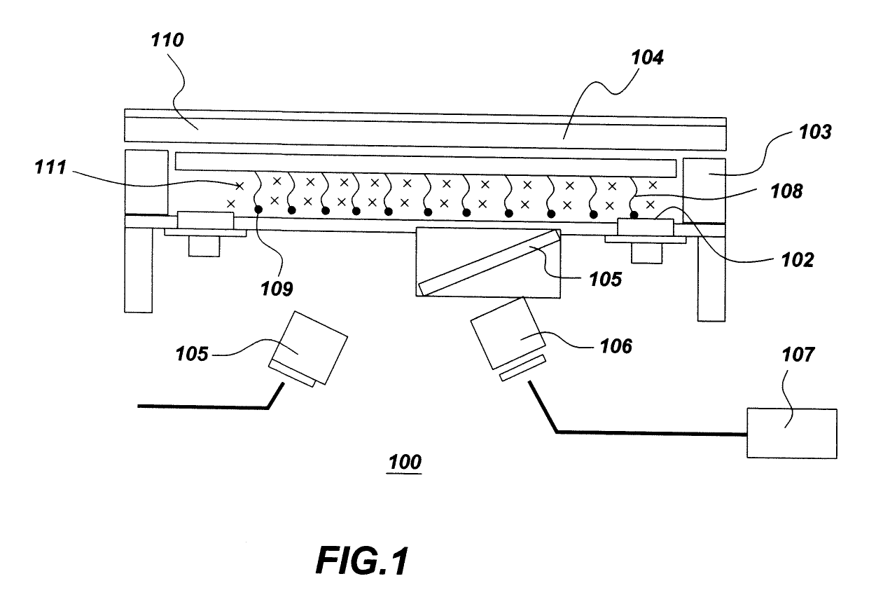

FIG. 1 illustrates one embodiment of an optical device 100 that incorporates aspects of the present invention. The optical device 100 includes a light source 101, a microfluidic chamber 102 consisting of a substrate 104, held in position by a support 103, a filter set 105, an optical detector 106, and a processor 107. A probe of interest 108 is attached directly through chemisorption to the surface of the substrate or the probe may be attached to the surface via a coating, 110, such as a thin layer of gold that facilitates attachment of the probe. Attached to the probe is a fluorophore 109 that absorbs light from the excitation source and emits light in the fluorescent range.

Referring further to FIG. 1 a sample of the analyte 111 is disposed within the mircofluidic chamber 102 and light is directed onto the sample. When the analyte 111 and the probe 108 having an attached fluorophores 109, interact to form a complex, the probe will emit light or will emit light that differs detectable from the light that they emit when the analyte is not bound to the probe. The emitted light passes through the filter set 105 and is detected by the optical detector 106. The filter is removed and the optical detector 106 detects reflected light. A processor 107 is coupled to the optical detector 106, and is configured to receive data from the optical detector. The processor 107 is further configured to perform an analysis on the sample of analyte 111.

In another embodiment, the fluorophore 109 is free in the sample solution. When the probe 108 and the analyte 111 interact with each other to form a probe-analyte complex, the fluorophore then associates with the complex, which alters its fluorescent profile such that it becomes fluorescent.

In an embodiment, the light source 101 is blue light from a LED or a blue laser diode which is directed onto the analyte sample contained in a microfluidic chamber 102 consisting of a glass cover 103 and a substrate 104 coated with a gold film 110. Attached to the gold surface is a probe containing ssDNA of a sequence of interest 108.

A solution containing buffer, the analyte 111, and SYBR Green I dye is pumped in to the microfluidic chamber 102 and allowed to react. If hybridization of the sample occurs with the capture ssDNA, the SYBR Green I binds to the newly formed dsDNA and fluoresces. The emitted light is passed through a band path filter set 105, which filters emissions greater than 525 nm. The band path filter set 105 is subsequently removed and reflected light is detected over the entire wavelength range of 300 to 700 nm. Alternatively a long band filter can be used or a filter that is capable of transmitting reflected light. Spectral data is transferred to the processor. This may be accomplished for example through an analog-to-digital converter.

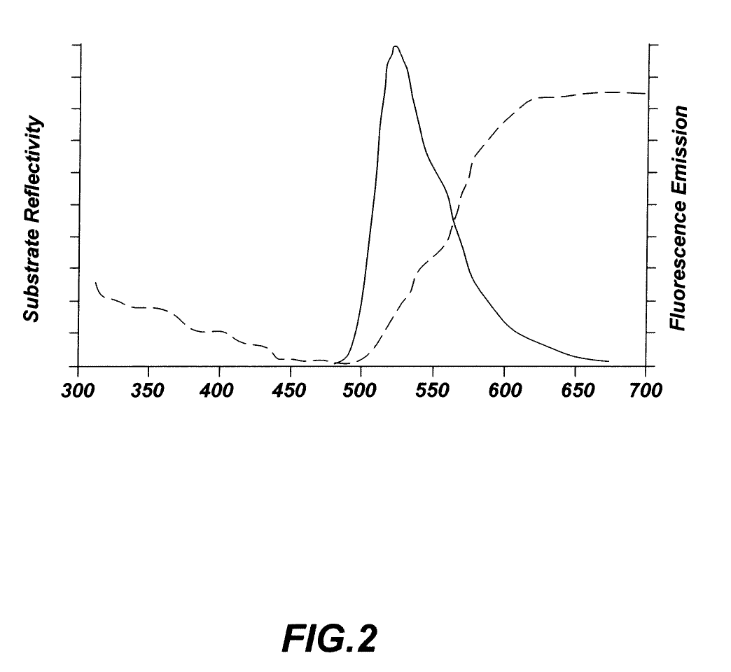

A representative spectral profile is shown in FIG. 2. The solid line within the graph represents the emission that will be provided by the bound SYBR Green I and allowed to pass to the photodiode 106. The dashed line in FIG. 2 shows the gold film reflectivity at a 30° angle as measured by the photo diode upon removal of the band path filter. The spectral data is analyzed and corrected for AR effect over the entire spectral range.

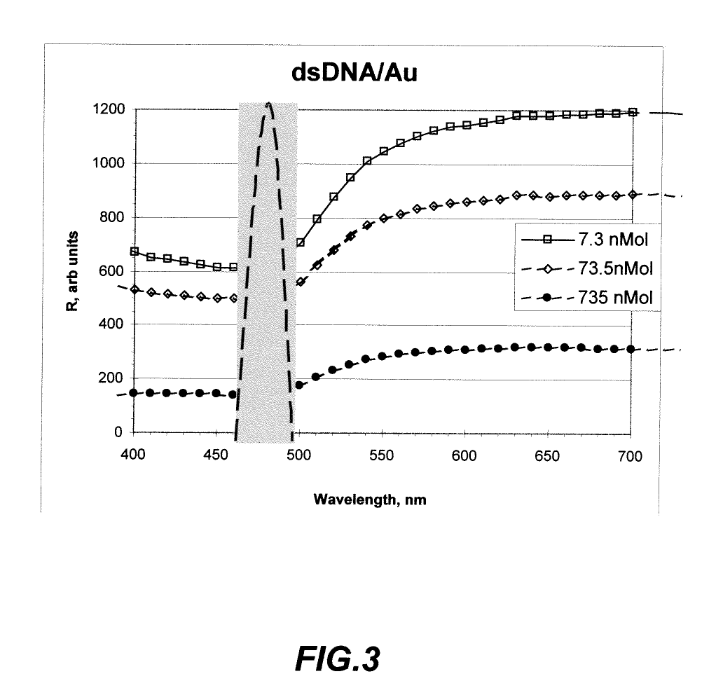

Experimental data depicting reflected light dependence as a function of captured analyte concentration is shown in FIG. 3. The reflectivity of the gold film substrate at different analyte concentration was measured using a spectro-goniometer under the 30° illumination and 25° collection angle. A blue LED spectrum is shown as a reference. Noticeable are the changes due to the dsDNA surface layer on the reflected light within the SYBR Green 1 excitation band (blue light, 400-500 nm) as well as within the emission band (green light, 520-565 nm). The former will introduce variation to the background noise at the detector level and contribute to secondary fluorescence of the bound dsDNA, while the latter will enhance fluorescence.

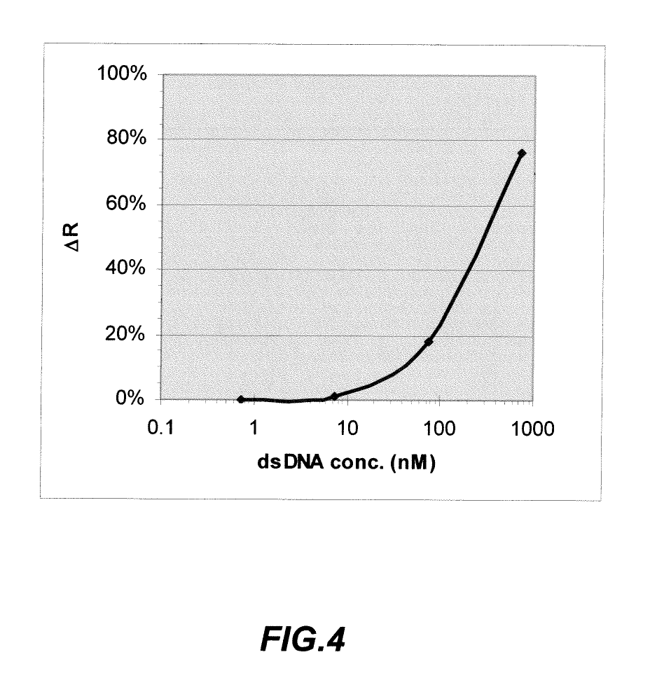

Since the AR signal also carries information about analyte properties, in some embodiments of the invention, AR may be further analyzed in juxtaposition with the fluorescent signal for greater accuracy of quantification. For example, the AR signal can be used to correct for variability in the fluorescent channel that may have been induced when the fluorescent dye is photo-bleached. AR may also be used to calculate concentration of analyte using AR calibration curves as shown in FIG. 4.

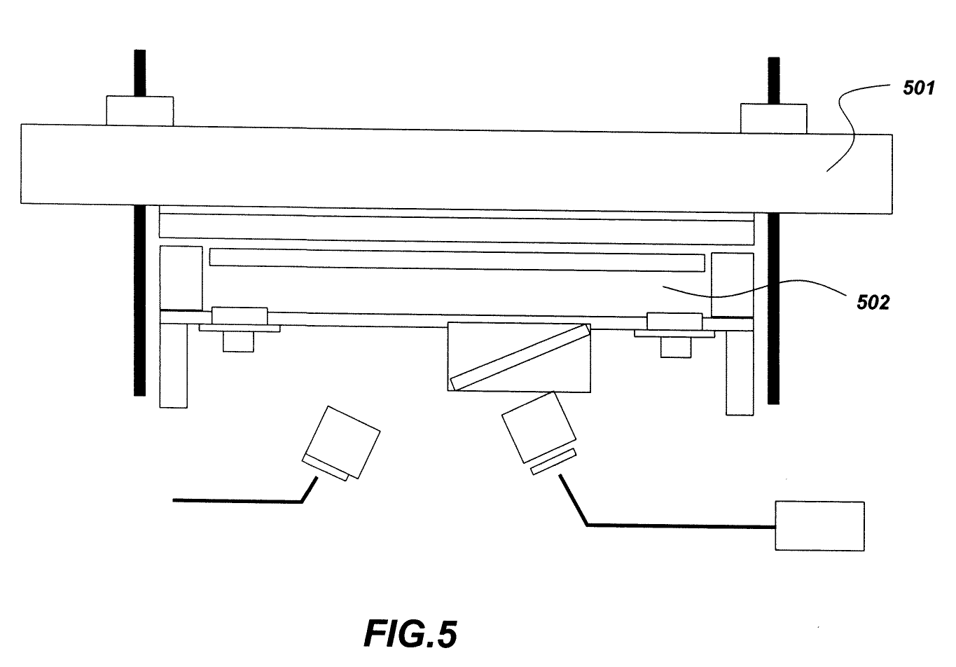

In one embodiment of the invention, fluorescent thermal de-annealing analysis of ds-DNA upon a gold substrate or “melt curve analysis” is improved by real-time deconvolution of change in fluorescence due to the coverage dependent change in the reflectivity of the gold and the change in fluorescence due to loss of ds-DNA by thermal de-annealing; either washing away of the fluorescent species or reduction in the fluorescent cross-section of an intercalation dye. Without applying this invention, the amount of DNA deannealed from the substrate would have to be quantified by first determining the change in reflectivity as a function of the deannealing and then correcting these reflectivity changes when analyzing the loss of fluorescence due to deannealing. As shown in FIG. 5 a micro-heater 501 is attached to the microfluidic chamber 502 and provides control over the thermal profile of the components of the chamber.

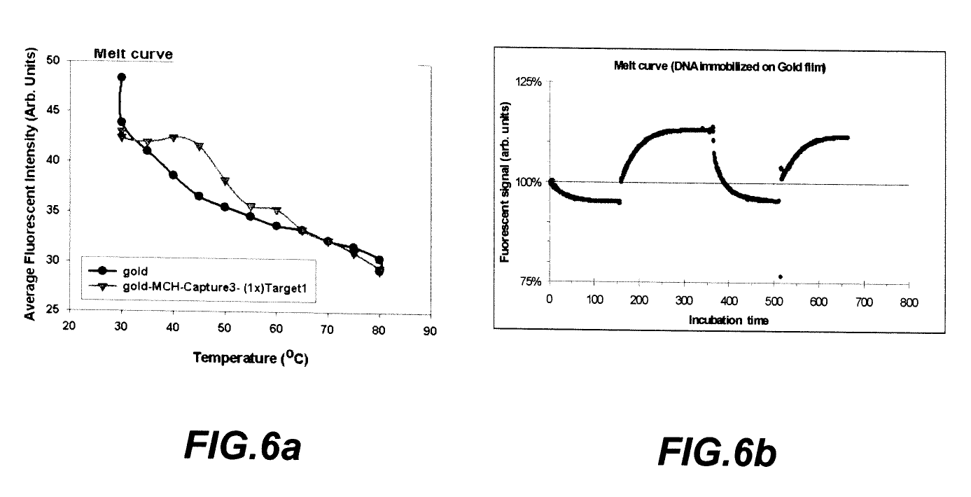

FIG. 6a shows the results of measuring the fluorescence of a captured analyte target with SYBR Green I during a linear temperature transition from 30° C. to 80° C. at 1.0° C./minute. Fluorescence data was converted into melting curves as shown in FIG. 6b. This is advantageous in applications dependent on the accuracy of fluorescence melting curves such as real time polymerase chain reaction analysis, monitoring of DNA amplification procedures, and detection of chromosomal translocation.

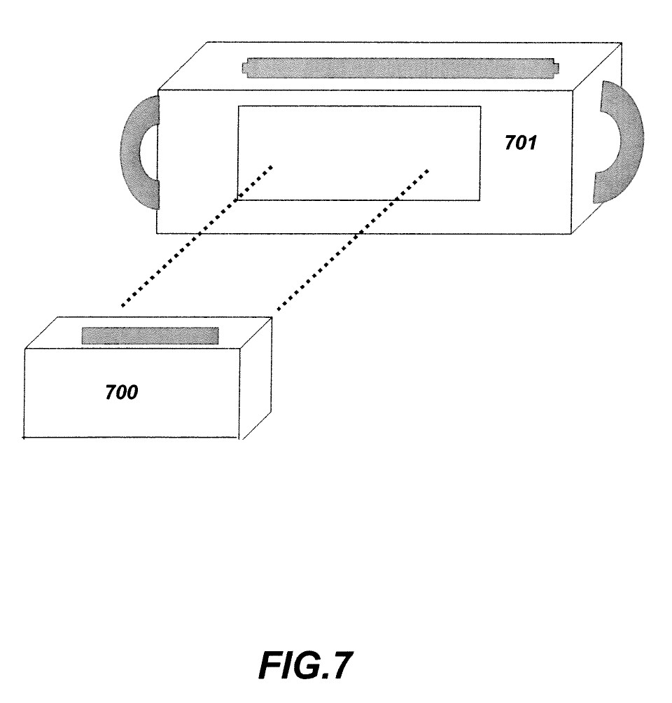

In another embodiment, the microfluidic chamber is removable to improve portability of the device. Alternatively, the binding surface itself may be removable. These embodiments enable off-site sample collection, sequential measurements, and facilitate disposal. FIG. 7 illustrates such a device wherein a removable chamber 700 is a component of a portable device 701 such as a hand held system. These embodiments are useful in applications requiring field-testing such as for the rapid detection of pathogens or chemical agents; for example, a confirmatory assay for the rapid identification of Legionella pneumophila and Legionella sppt.

Although the preceding examples are for detection and quantitative measurement of DNA, the invention is applicable to other applications. For example the invention is applicable to other types of nucleic acid recognition (e.g., RNA, LNA, PNA, aptamer), peptide recognition (e.g., zinc-fingers), protein recognition (e.g., avidin/biotin, antibodies and all known fragments, enzyme), and chemical recognition (e.g., ligands, crown-ethers, cyclodextran).

While only certain features of the invention have been illustrated and described herein, many modifications and changes will occur to those skilled in the art. It is, therefore, to be understood that the appended claims are intended to cover all such modifications and changes as fall within the true spirit of the invention.

1. A device for detecting one or more analytes bound directly or indirectly to a binding substrate functionalized with a fluorescent probe specific for the one or more analytes, comprising:
an excitation source for illuminating the binding substrate;
a filter capable of transmitting fluorescent light and a filter capable of transmitting reflected light emitted from the binding substrate;
a detector adapted to detect the fluorescent light and the reflected light emitted from the binding substrate; and
a processor in communication with the detector and adapted to determine whether one or more of the analytes is bound to the binding substrate by correlating the emitted fluorescent light with the reflected light.

2. The device of claim 1 wherein one or more of the analytes are pathogens.

3. The device of claim 1 wherein one or more of the analytes comprises proteins or nucleic acids.

4. The device of claim 1 wherein the binding substrate further comprises a coating to facilitate binding one or more of the analytes directly or indirectly to the binding substrate.

5. The device of claim 4 wherein the coating comprises gold.

6. The device of claim 1 wherein the excitation source and the optical detector are positioned on the same side of the substrate and orientated at predetermined angles.

7. The device of claim 1 wherein the excitation source comprises a light emitting diode.

8. The device of claim 7 wherein the light emitting diode is a blue laser diode.

9. The device of claim 1 wherein the detector is a photodiode or a photomultiplier tube.

10. The device of claim 1 wherein one or more of the filters capable of transmitting fluorescent light and capable of transmitting reflected light is interchangeable.

11. The device of claim 1 wherein the filter capable of transmitting fluorescent light is a band-pass filter or a long-pass filter.

12. The device of claim 1 further comprising an optical filter positioned between the excitation source and the binding substrate.

13. The device of claim 12 wherein the optical filter is a band-pass filter.

14. The device of claim 1, further comprising a compartment to house the binding substrate.

15. The device of claim 14 wherein the compartment is a removable cartridge.

16. The device of claim 15 wherein the removable cartridge is disposable.

17. The device of claim 14 further comprising a temperature controller for controlling the temperature of the compartment.

18. The device of claim 17 wherein the temperature controller is a micro-heater capable of holding the compartment.

19. A method for detecting one or more analytes bound directly or indirectly to a binding substrate functionalized with a fluorescent probe specific for the one or more analytes comprising:
illuminating the binding substrate;
measuring any fluorescent light and reflected light emitted from the binding substrate; and
determining whether one or more of the analytes is bound to the binding substrate by correlating the measurements of emitted fluorescent light and reflected light.

20. The method of claim 19 wherein the measuring step is performed by measuring the reflected light and fluorescent light simultaneously.

21. The method of claim 19 wherein the measuring step is performed by measuring the reflected light and the fluorescent light sequentially.

22. The method of claim 19 wherein the correlating step comprises;
analyzing the measurements of fluorescent light and reflected light to identify variations induced by reflectivity; and
generating a fluorescent spectral reading based at least in part on the one or more analytes by correcting for the variations.

23. The method of claim 19 wherein the binding substrate further comprises a coating to facilitate attachment of the fluorescent probe.

24. The method of claim 23 wherein the coating comprises gold.
